(19) United States
(12) Patent Application Publication
(10) Pub. No.: US 2005/0202075 A1
(43) Pub. Date: Sep. 15, 2005

(54) Delivery of genes encoding short hairpin RNA using receptor-specific nanocontainers

(76) Inventors: William M. Pardridge, Los Angeles, CA (US); Ruben J. Boado, Los Angeles, CA (US)

Correspondence Address: DAVID J. OLDENKAMP, ESQ., SHAPIRO & DUPONT LLP, 233 WILSHIRE BOULEVARD, SUITE 700, SANTA MONICA, CA, 90401, US

(21) Appl. No.: 10/800,362

(22) Filed: Mar. 12, 2004

(51) Int. Cl. A61K 48/00; A61K 9/127; C12N 15/88

(52) U.S. Cl. 424/450; 514/44; 435/458

(57) ABSTRACT

Receptor-specific nanocontainers are used to deliver a gene that encodes short hairpin RNA to cells having a given receptor. Once inside the cell, the gene expresses short hairpin RNA that includes a nucleotide sequence that is antisense to at least a portion of an oncogene, such as human epidermal growth factor receptor (EGFR) mRNA, or other disease causing nucleotide sequence. The short hairpin RNA is converted, in the cellular cytoplasm, into short RNA duplexes that are effective in deactivating (knocking down) the oncogenic or disease causing gene.

### BACKGROUND OF THE INVENTION

1. Field of the Invention

The present invention relates generally to the delivery of gene medicines to organs and tissues within the body including the brain. More particularly the present invention involves antisense gene therapy using a combination of liposome technology, receptor technology, pegylation technology and therapeutic gene technology. The invention provides formulations that are useful in treating brain cancer and other solid cancers.

2. Description of Related Art

The publications and other reference materials referred to herein to describe the background of the invention and to provide additional detail regarding its practice are hereby incorporated by reference. For convenience, the reference materials are numerically referenced and grouped in the appended bibliography.

Gene therapy has been used in over 5000 patients in more than the last 10 years, with no success in the treatment of cancer in humans, including brain cancer (1). While there has been much success in cancer therapeutics in Petri dishes and rudimentary animal models, this progress has not been translated to humans with cancer. The inability to jump from Petri dishes to people arises from the severe gene delivery barriers in the body in humans, which are non-existent in cell culture systems. With respect to gene therapy of brain cancer, the therapeutic gene has been incorporated in viral vectors and injected into the brain following craniotomy (1). However, this approach is defective for two reasons. First, the only way to deliver the therapeutic gene to all cancer cells in the brain is to deliver the gene across the vascular barrier of the tumor, which forms the blood-brain barrier (BBB). Drilling a hole in the patient's head and injecting the gene via this hole only delivers the gene to a small percentage of cancer cells (1). Second, the use of viral vectors is a problem.

Viral vectors such as adenovirus or herpes virus cause inflammation in the brain leading to demyelination (2,3). Viral vectors such as retrovirus or adeno-associated virus (AAV) cause random and permanent integration in the patient's chromosomes (4,5), which can lead to cancer secondary to insertional mutagenesis. Therefore, the limiting factor in gene therapy is delivery, both with respect to the need to have a non-viral delivery system, and to the need to have a gene delivery system that crosses the BBB following an intravenous injection. Such a gene delivery system is taught in U.S. Pat. No. 6,372,250 wherein pegylated immunolipsomes (PILS) are used to deliver genes to tissues and organs, including the brain.

A special type of gene therapy that aims to knock out the expression of a pathologic gene is called antisense gene therapy. The therapeutic gene encodes a long strand of RNA that is antisense to the target mRNA in the cell. The antisense RNA forms a duplex with the target mRNA, and this leads to either degradation of the target mRNA or arrest of the mRNA translation, which causes a form of post-transcriptional gene silencing (PTGS). Within the last few years, it has been discovered that PTGS can be caused by the injection into the cell of short RNA duplexes of approximately 20 nucleotides in length (6).

The RNA duplex has a defined sequence that is antisense to the target mRNA, and the formation of the complex between the RNA duplex and the target mRNA leads to either degradation of the target mRNA or mRNA translation arrest, and PTGS. This form of PTGS is called RNA interference (RNAi), because it is mediated by a short RNA duplex (7). Because RNA is very unstable in vivo, a DNA-based form of RNAi has been developed (6), wherein an expression plasmid encodes a short hairpin RNA (shRNA), which is comprised of a stem-loop structure. Once the shRNA is expressed in the cell, it is transported to the cytoplasm and processed into a short RNA duplex, which can then cause PTGS of the target mRNA (6). The shRNA may be 100% complementary to the target mRNA, and act as a silencing RNA (siRNA) to cause target mRNA degradation. Alternatively, the shRNA may have an incomplete complementarity to the target mRNA sequence, and act as a micro RNA (mRNA) to cause target mRNA translation arrest. While RNAi holds much promise for the treatment of cancer, viral infections, and other diseases, the application of RNAi in humans is still another form of gene therapy, and, as such, is limited by the same delivery problems as any other form of gene therapy (8). Since there has been no clinical success in humans of any form of gene therapy, there is little reason to believe that RNAi-based gene therapy will be successful with the standard approaches used in the past.

The epidermal growth factor receptor (EGFR) is over-expressed in 90% of primary highly malignant brain cancer, called glioblastoma multiforme (GBM) (9), and is over-expressed in 70% of solid cancer, in general (10). The EGFR plays a tumorigenic role in these cancers, and is currently the most intensively studied target in the development of new cancer therapeutics. A variety of approaches have been tried to ‘knock down’ the EGFR in cancer, including small molecules (11), and EGFR specific monoclonal antibodies (12). Although research in Petri dishes has shown that targeting the EGFR with antisense gene therapy is feasible (13), there had been no reduction to practice of this approach in living animals with cancers, including brain cancer, owing to the inability to solve the delivery problem.

A non-viral expression plasmid has been produced that encodes for a 700 nucleotide (nt) RNA that was antisense to the human EGFR mRNA around nt 2300-3000 (14). This expression plasmid was delivered to mice with brain cancer using the PIL gene targeting approach, and a 100% increase in survival time of the mice was achieved (15). However, in order to increase the potency of this expression plasmid, it was necessary to include in the plasmid the gene encoding the Epstein Barr nuclear antigen (EBNA)-1 (15). EBNA-1 increases gene expression in dividing cells, and resulted in a 10-fold increase in expression of exogenous genes in human brain cancer cells following delivery to the cell with the PIL gene targeting technology (16). However, EBNA-1 is tumorigenic (17), and could lead to cancer if included in a gene used in humans. Therefore, what is needed is a new more potent form of antisense gene therapy that can be directed at the EGFR and that does not require the use of EBNA-1 to achieve the desired therapeutic effect.

One report shows that PTGS of the human EGFR can be achieved in cell culture with synthetic RNA duplexes delivered with a cationic lipid (18). However, no prior work has demonstrated that it would be possible to cause PTGS of the human EGFR with an shRNA that was produced within the cell from an shRNA expressing plasmid DNA, i.e., DNA-based RNAi. Augmenting the uncertainty as to whether it would be possible to knock down the EGFR with DNA-based RNAi is the fact that, in general, it is difficult to find a target sequence within any mRNA that yields a full RNAi effect, and it is generally regarded that only 20% of all sequences tested will be effective (6).

Prior work had shown that a plasmid DNA encoding an shRNA against the luciferase gene could be delivered to adult rats with brain cancer using the PIL gene targeting approach (19). However, since the luciferase gene expressed in this form of brain cancer was only a reporter gene, it was not possible to evaluate whether the delivery of RNAi-encoding genes to brain cancer with the PIL gene targeting technology could cause any benefit on survival.

### SUMMARY OF THE INVENTION

In accordance with the present invention, receptor-specific nanocontainers are used to deliver short hairpin RNA genes into cells that have a given receptor. Once inside the cell, the gene expresses short hairpin RNA that includes a nucleotide sequence that is antisense to at least a portion of an oncogenic gene, such as human epidermal growth factor receptor (EGFR) mRNA, or other disease causing gene. The short hairpin RNA is converted, in the cellular cytoplasm, into short RNA duplexes that are effective in deactivating (knocking down) the oncogenic or disease causing gene.

It was discovered that certain regions of oncogenic genes, such as EGFR mRNA, are more susceptible to attack using the receptor-specific nanocotainers of the present invention. For example, it was found that genes expressing short hairpin RNA that is antisense to the portion of EGFR mRNA located between numbered nucleotides 2300 and 3800 are effective in treating cancer. It was further found that genes expressing short hairpin RNA that is antisense to the portion of EGFR mRNA located between numbered nucleotides 2500 and 3000 are particularly effective in treating cancer. The portion of the EGFR mRNA gene located between numbered nucleotides 2500 and 2600 was found to be especially susceptible to attack by short hairpin RNA in accordance with the present invention.

The present invention also covers methods for delivering short hairpin RNA to cells having a receptor. The methods include the step of administering to an animal an effective amount of a preparation that includes receptor-specific nanocontainers that contain the plasmid DNA encoding the shRNA in accordance with the present invention and a pharmaceutically acceptable carrier for the receptor-specific nanocontainers. The preparation is administered by way of a non-invasive procedure, such as intravenous injection. In survival studies conducted using weekly intravenous RNAi gene therapy in accordance with the present invention, a significant increase in survival time in adult mice with intra-cranial human brain cancer was observed.

The above discussed and many other features and attendant advantages of the present invention will become better understood by reference to the detailed description when taken in conjunction with the accompanying drawings.

### BRIEF DESCRIPTION OF THE DRAWINGS

FIG. 1A is a diagrammatic representation of an exemplary pegylated immunoliposome (PIL) in accordance with the present invention. The liposome surface is conjugated with several thousand conjugating agents such as 2000 Dalton polyethylene glycol (PEG), which are depicted as strands projecting from the surface. The tips of about 1-2% of the PEG strands are conjugated with a targeting ligand comprised of either the 8D3 rat monoclonal antibody to the mouse transferrin receptor (mTfR) (MAb1) and the murine 83-14 monoclonal antibody to the human insulin receptor (HIR) (MAb2). Encapsulated in the interior of the PIL is the plasmid DNA encoding the short hairpin RNA (shRNA) that produces the RNA interference (RNAi). The gene encoding the shRNA is driven by the U6 promoter (pro) and is followed on the 3′-end with the T5 termination sequence for the U6 RNA polymerase. (B) The nucleotide sequence of the human epidermal growth factor receptor (hEGFR) sequence between nucleotides 2529-2557 is shown on top (SEQ.ID.NO. 1), which is derived from the Genbank deposited sequence for the human EGFR (accession number X00588). The sequence and secondary structure of the shRNA produced by clone 967 is shown on the bottom of FIG. 1B (SEQ. ID. NO. 2). The antisense strand is 5′ to the 8-nucleotide loop, and the sense strand is 3′ to the loop. The sense strand contains 4 G/U mismatches to reduce the Tm of hybridization of the stem loop structure; the sequence of the antisense strand is 100% complementary to the target mRNA sequence. FIG. 1C shows the results of tests wherein human U87 glioma cells were incubated with [³H]-thymidine for a 48 hr period that follows a 5 day period of incubation of the cells with HIRMAb-targeted PILs carrying either clone 967 or 882 plasmid DNA. A dose of 1.4, 14, 140, or 1400 ng plasmid DNA per dish was used in each experiment. Data are mean±SE (n=3 dishes).

FIG. 2 depicts the results of survival study in which intravenous RNAi gene therapy directed at the human EGFR in accordance with the present invention was initiated at 5 days after implantation of 500,000 U87 cells in the caudate putamen nucleus of scid mice. Weekly intravenous gene therapy was repeated at days 12, 19, and 26 (arrows). The control group was treated with saline on the same days. There were 11 mice in each of the 2 treatment groups. The time at which 50% of the mice were dead (ED₅₀) is 17 days and 32 days in the saline and RNAi groups, respectively. The RNAi gene therapy using short hairpin antisense RNA in accordance with the present invention produced an 88% increase in survival time, which is significant at the p<0.005 level (Fisher's exact test).

FIG. 3 depicts the results of immunocytochemistry studies in which mouse brain autopsy sections were stained with either the rat 8D3 MAb to the mouse TfR (panels A-E) or rat IgG (panel F). No sections were counterstained. The magnification in panels A, B, D, E, and F is the same and the magnification bar in panel A is 135 μm. The magnification bar in panel C is 34 μm. Panels A-C are sections taken from the brain of the saline treated mice, and panels D-F are sections of brain taken from mice treated with the clone 967 gene therapy. Panels A-C show the density of the tumor vasculature in the saline treated mice. Panel B shows a section containing normal brain at the bottom of the panel and tumor at the top of the panel; the tumor is vascularized by a vessel originating from normal brain. Panel D shows the tumor on the left of the panel and normal brain on the right side of the panel; this section is taken from a mouse treated with RNAi gene therapy, and illustrates the decreased vascular density in the RNAi treated animals. The vascular density of normal brain is not changed in the RNAi treated animals as shown in panel E.

FIG. 4 depicts the results of in vivo EGFR down-regulation by RNAi gene therapy in accordance with the present invention. Confocal microscopy of intra-cranial glioma sections are shown for brain tumors from RNAi treated mice (A-C) or saline treated mice (D-F). The sections are doubly labeled with the murine 83-14 MAb to the HIR (green) and the rat 8D3 MAb to the mouse TfR (red). There is decreased immunoreactive EGFR in the tumor cells in the RNAi treated mice (A-C) relative to the saline treated mice (D-F). The saline treated animals died at 14-15 days post-implantation (D, E, and F), whereas the RNAi-treated animals died at 31 days (A), 33 days (B), and 34 days (C) post-implantation, respectively, which was 5, 7, and 8 days following the last dose of intravenous RNAi gene therapy (FIG. 2).

FIG. 5 shows the selective knockdown of the immunoreactive EGFR in human U87 cells exposed to either clone 967 or clone 882, but not by clone 952 or clone 962, and determined by Western blotting. These Western blot studies in cell culture corroborate the confocal microscopy of the in vivo brain tumor results shown in FIG. 4.

### DETAILED DESCRIPTION OF THE INVENTION

The present invention provides compositions and methods that are useful to inactivate (“knock down”) pathologic genes in animals using a combination of gene therapy, RNA interference (RNAi) using short hairpin antisense RNA and gene targeting technology. The invention is based on the gene targeting technology described in U.S. Pat. No. 6,372,250, which teaches methods and compositions for non-invasive, non-viral delivery of therapeutic genes. This technology enables the targeting of therapeutic genes to distant sites following a simple intravenous injection of a non-viral formulation of the gene medicine. The combination of this gene targeting technology and the methods of RNAi, which is a form of antisense gene therapy, enables the knockdown in animals of disease causing genes.

The receptor-specific nanocontainers of the present invention are designed for delivering short hairpin RNA to a cell having a receptor. The composition includes a nanocontainer that has an exterior surface and an internal compartment. A plurality of receptor targeting agents are attached to the surface of the nanocontainer by way of conjugation agents. The targeting agents provide the nanocontainer with its receptor-specific targeting capability. A gene is located within the internal compartment of the nanocontainer. The gene includes a sufficient amount of genetic information to encode a short hairpin RNA. The nucleotide sequence of the short hairpin RNA includes nucleotides that are antisense to at least a portion of mRNA or other nucleotide sequence that is necessary for the receptor-targeted cell to function.

The nanocontainer is preferably a liposome, but may be any other suitable nanocontainer that includes an exterior surface and an internal compartment for housing the short hairpin RNA. The liposomes preferably have diameters of less than 200 nanometers. Liposomes having diameters of between 50 and 150 nanometers are preferred. Especially preferred are liposomes or other nanocontainers having external diameters of about 80 to 100 nanometers. Suitable types of liposomes are made with neutral phospholipids such as 1-palmitoyl-2-oleoyl-sn-glycerol-3-phospho-choline (POPC), diphosphatidyl phosphocholine, distearoylphosphatidylethanolamine (DSPE), or cholesterol, along with a small amount (1-5%) of cationic lipid, such as didodecyldimethylammonium bromide (DDAB) to stabilize the anionic DNA within the liposome. The procedures for preparing and using liposomes as nanoconatainers for non-invasive gene targeting are well known, as taught in U.S. Pat. No. 6,372,250 and copending U.S. application Ser. Nos. 10/025,732 and 10/647,197.

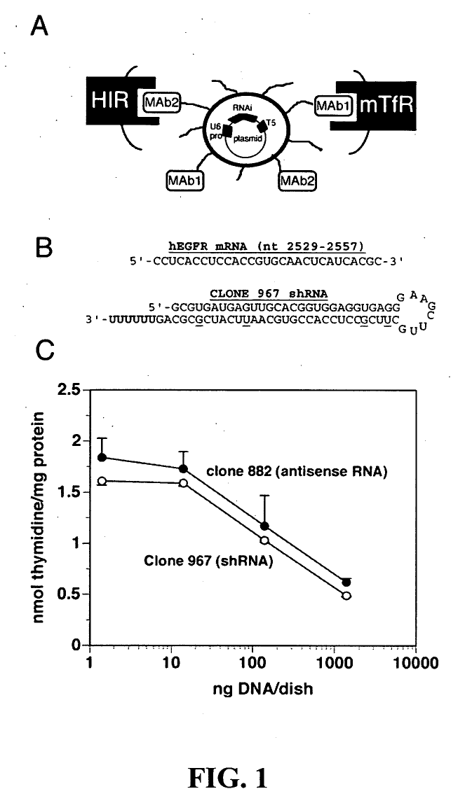

The gene which is encapsulated within the liposome or other nanocontainer can be any gene that encodes a short hairpin RNA (shRNA) that includes a sufficient amount of an antisense sequence to deactivate or at least attenuate the target mRNA or other nucleotide sequence. For the purposes of this specification “short hairpin RNA” is RNA that has a stem length from 19 to 29 nucleotides, a loop length of 5-10 nucleotides and has the hairpin shape as shown in FIG. 1B. The short hairpin RNA should contain an antisense portion of the short hairpin RNA and should be from 19 to 29 nucleotides long and may vary depending upon the size of the accessible site within the target mRNA. Exemplary short hairpin RNA that may be used include those that are antisense to oncogenic receptors such as the EGFR or ras, or to angiogenic factor or receptors, such as the vascular endothelial growth factor (VEGF) or the VEGF receptor (VEGFR). It is preferred that the gene that encodes the short hairpin RNA be expressed by plasmid DNA that is encapsulated within the internal compartment of the liposome or nanocontainer.

The short hairpin gene may be encapsulated within the liposome according to any of the well-known drug encapsulation processes. For example, encapsulation may be accomplished by sonication, freeze/thaw, evaporation, detergent dialysis, and extrusion through membrane filters.

The number of genes encapsulated within the liposome mixture may vary from 1 to many, depending on the disease being treated, although each individual nanocontainer may carry no more than 1 or 2 plasmid DNA molecules depending on the effective radius of the plasmid DNA. The limiting factors will be the size of the gene that is being encapsulated and the size of the internal compartment of the liposome. Using polycationic proteins such as histone, protamine, or polylysine, it is possible to compact the size of plasmid DNA that contains several thousand nucleotides to a structure that has a diameter of 10-30 nm. The genes used to express the short hairpin RNA are relatively small so that many genes may be incorporated in a single tandem expression plasmid DNA. If desired, it is possible to encapsulate many copies of the same gene or multiple copies of multiple genes within the expression plasmid DNA, prior to encapsulation of the DNA inside the liposome. In general, it is desirable to maximize the number of genes present in any given nanocontainer as much as possible.

In order to provide transport of the encapsulated gene to the desired target cell (and across the blood-brain barrier, if necessary), a number of targeting agents are conjugated to the surface of the nanocontainer. Suitable targeting agents include any agent that is able to target the nanocontainer to the desired receptors located on the cell surface. Although any number of targeting agents may be used, preferred agents include—endogenous receptor ligands, such as insulin, transferrin, insulin-like growth factors, leptin, fibroblast growth factors, or peptidomimetic monoclonal antibodies (MAb) that, like the endogenous ligand, also bind the receptor. Either the endogenous ligand or the peptidomimetic MAb must be an endocytosing ligand, such that receptor binding on the external surface of the cell is followed by receptor-mediated endocytosis into the interior of the cell. In general, the targeting ligand must inititate endocytosis of the liposome across the cell membrane of both the vascular endothelial cell and the target tumor cell behind the vascular barrier. Targeting agents that are able to target the liposome across both the vascular endothelial membrane barrier and the target cell membrane barrier are preferred. For this to happen the targeted receptor would have to be expressed on both the vascular endothelial barrier and the target cell membrane. In the case of brain, the vascular endothelial cell membrane is the BBB. Such targeting agents, or “transportable peptides,” include insulin, transferrin, insulin-like growth factor, or leptin, or their corresponding peptidomimetic MAb's, as all target cognate receptors that are expressed on both the BBB and on the brain cell membrane (BCM). Alternatively, the surface of the liposome can be conjugated with two different “transportable peptides,” one peptide targeting an endogenous BBB receptor and the other targeting an endogenous BCM peptide. The latter could be specific for particular cells within the brain, such as neurons, glial cells, pericytes, smooth muscle cells, or microglia. Targeting peptides may be endogenous peptide ligands of the receptors, analogues of the endogenous ligand, or peptidomimetic MAbs that bind the same receptor of the endogenous ligand. The use of transportable peptides, in general, and the use of transferrin or insulin as a targeting ligand is described in detail in U.S. Pat. No. 4,801,575. Receptor (TfR)-specific peptidomimetic monoclonal antibodies as BBB “transportable peptides” are described in detail in U.S. Pat. Nos. 5,154,924; 5,182,107; 5,527,527; 5,672,683; 5,833,988; and 5,977,307. The use of a MAb to the human insulin receptor (HIR) as a BBB “transportable peptide” is preferred. Exemplary preferred MAb's to the human insulin receptor are disclosed in U.S. patent application Ser. No. 10/307,276.

The conjugation agents that are used to conjugate the targeting agents to the surface of the liposome can be any of the well-known polymeric conjugation agents such as sphingomyelin, polyethylene glycol (PEG) or other organic polymers. PEG is an especially preferred conjugation agent. The molecular weight of the conjugation agent is preferably between 1000 and 50,000 DA. A particularly preferred conjugation agent is a bifunctional 2000 DA PEG that contains a lipid at one end and a maleimide group at the other end. The lipid end of the PEG inserts into the surface of the liposome, whereas the maleimide group forms a covalent bond with the receptor-specific monoclonal antibody or other blood-brain barrier targeting vehicle. It is preferred that from 5 to 1000 targeting vehicles be conjugated to each liposome. Liposomes having approximately 25-75 targeting vehicles conjugated thereto are particularly preferred.

Although liposomes are the preferred nanocontainer, it will be recognized by those skilled in the art that other nanocontainers may be used. For example, the liposome can be replaced with a nanoparticle or any other molecular nanocontainer with a diameter <200 nm that can encapsulate the gene and protect the nucleic acid from nucleases while the formulation is still in the blood or in transit from the blood to the intracellular compartment of the target cell. Also, the PEG strands can be replaced with multiple other polymeric substances such as sphingomylein, which are attached to the surface of the liposome or nanocontainer and serve the dual purpose of providing a scaffold for conjugation of the “transportable peptide” and for delaying the removal of the formulation from blood and optimizing the plasma pharmacokinetics. Further, the present invention contemplates delivery of genes expressing short hairpin antisense RNA to a variety of cells or organs which have specific target receptors, including brain, liver, lung, and spleen. In addition, the present invention contemplates the delivery of shRNA expressing genes across the blood-retinal barrier to the retina and other ocular structures, as described in detail in copending U.S. application Ser. No. 10/025,732. The receptor-specific nanocontainers in accordance with the present invention may be combined with any suitable pharmaceutical carrier for intravenous administration. Intravenous administration of the receptor-specific nanocontainers is the preferred route since it is the least invasive. Other routes of administration are possible, if desired. Suitable pharmaceutically acceptable carriers include saline, Tris buffer, phosphate buffer, or any other aqueous solution.

A therapeutically effective amount of the receptor-specific nanocontainers will vary widely depending upon the individual being treated and the particular gene being administered. The appropriate dose will be established by procedures well known to those of ordinary skill in the art.

Brain cancer, and most solid cancers in general, over-express the epidermal growth factor receptor (EGFR). The EGFR plays a tumorigenic role in these cancers. Many current cancer treatments are aimed at inhibiting the EGFR. The following description of preferred exemplary embodiments of the present invention demonstrate how the EGFR can be knocked out in brain cancer in vivo with non-invasive gene therapy that does not use viral vectors and requires a simple intravenous administration.

The exemplary target gene described in the examples of the following detailed description is the human epidermal growth factor receptor (EGFR), which plays a tumorigenic role in brain cancer (20,21) and in the majority of solid cancers in general (10). In this description a mouse model of human intra-cranial brain cancer is used to demonstrate the ability of the present invention is to prolong survival in cancer patients. It will be understood by those of ordinary skill in the art that the invention may also be used for knocking down other target genes that may be involved in cancer or other disease. Other oncogenic causing genes that may be targeted using short hairpin RNA antisense treatment include mutants of the EGFR, wherein the oncogenic kinase domain is constitutively active, and which is expressed by mutant forms of the EGFR mRNA with sequences different from the wild type EGFR. Many brain cancers and other solid cancers express various EGFR mutants such as the vIII EGFR mutant. Other oncogenic gene targets which are receptors include HER2, HER3, HER4 in GBM, breast, ovary, lung, and head and neck cancer, and the fibrobalst growth factor receptor (FGFR) in lung, ovary, and breast cancer, the platelet derived growth factor receptor (PDGFR) in GBM, the insulin-like growth factor receptor-1 (IGFR1) in solid tumors (39). Other oncogenic gene targets which are growth factors include transforming growth factor-α (TGF-α) in cancers over-expressing the EGFR, PDGF in GBM, or VEGF to block angiogenesis in cancer (39). Other oncogenic gene targets that include altered protein kinases include the Bcr-Abl in chronic myelogenous leukemia (CML), c-Met in renal cancer, c-Kit in stomach cancer, ras in multiple cancers, raf in bladder, colon, lung, or breast cancer, or CdKs in multiple cancers (39). Non-cancer chronic disease that would benefit from the antisense knockdown of disease causing genes include viral infections such as chronic hepatitis or acquired immune deficiency syndrome (AIDS), where target genes are viral specific genes crucial to viral replication. The most common cause of age related blindness is age related macular degeneration or AMD, which is caused by vascular hypertropy induced by VEGF, and antisense knockdown of either the VEGF gene or the VEGF-R gene in the eye could provide new therapy for AMD (40).

As will be shown below, an exemplary embodiment of the present invention (shRNA expressed by clone 967) was used to achieve an 88% increase in survival time of adult mice with pre-formed intra-cranial human brain cancer following the weekly intravenous administration of clone 967 plasmid DNA and delivered to the brain cancer with the PIL gene targeting technology. Clone 967 is an exemplary eukaryotic expression plasmid that encodes an shRNA directed at nt 2529-2557 of the human EGFR. This type of shRNA may be used in treating brain cancer and in treating other solid cancers, in general. The increase in survival time was achieved without craniotomy or other invasive form of administration and required only simple weekly intravenous injections. The therapeutic effect is achieved without the use of viruses or tumorigenic DNA elements such as EBNA-1. This invention provides a combination of DNA-based RNAi technology and the PIL gene targeting technology that may be used to knock down cancer causing genes other than the EGFR in either primary brain cancer or in non-brain cancer that has metastasized to brain. In addition, the receptor-specific nanocontainer may be used to knock down disease causing genes in the brain for disorders other than cancer.

Gene therapy of brain cancer offers the promise of specifically knocking down the expression of oncogenic genes such as EGFR. However, gene therapy is limited by the delivery problem, which is particularly difficult in brain owing to the presence of the BBB. To circumvent the BBB, attempts have been made to deliver therapeutics to brain cancer by the craniotomy approach (1). However, this approach is not effective as there is very limited distribution of the therapeutic within the tumor following an intra-tumor injection (1). Therapeutics can be delivered to all cells in brain cancer via the transvascular route across the BBB (22). The transvascular delivery of non-viral genes to brain is now possible using a new non-viral gene transfer technology that uses pegylated immunoliposomes (PILs) as mention previously (see U.S. Pat. No. 6,372,250). With this approach, the non-viral plasmid DNA is encapsulated in the interior of an 85 nm anionic liposome, and the surface of the liposome is conjugated with several thousand strands of polyethylene glycol (PEG). This “PEGylation” process restricts uptake of the liposome by the reticulo-endothelial system, and enables a prolonged blood residence time (23). The PEGylated liposome is then targeted across biological barriers in vivo with receptor-specific peptidomimetic monoclonal antibodies (MAb) as depicted in FIG. 1A.

The application of the PIL non-viral gene transfer technology enabled a 100% increase in survival time of mice with intra-cranial human brain cancer with weekly intravenous injections of antisense gene therapy directed at the human EGFR (15). A eukaryotic expression plasmid, designated clone 882, that encodes for a 700 nucleotide RNA that is antisense to nucleotides 2317-3006 of the human EGFR (14), was encapsulated in PILs that were doubly targeted to brain cancer in vivo with 2 MAbs of different receptor specificities (15). One MAb, the rat 8D3 MAb to the mouse transferrin receptor (TfR), enabled transport of the PIL across the mouse BBB that vascularized the intracranial cancer; these cancer vessels were of mouse brain origin and expressed the mouse TfR. A second MAb targeted the human insulin receptor (HIR) that was expressed on the human brain cancer plasma membrane (FIG. 1A). The targeting MAbs act as molecular Trojan horses to ferry the PIL across membrane barriers, and these MAbs are species specific (24). The 8D3 to the mouse TfR enabled transport across the first barrier, the mouse BBB, but did not mediate transport of the PIL across the second barrier, the human brain cancer cell membrane. This was accomplished with the HIRMAb, which similarly, would not react with the mouse vascular endothelial insulin receptor. The doubly conjugated PIL is designated HIRMAb/TfRMAb-PIL (FIG. 1A).

In order to augment the potency of the clone 882 expression plasmid, this vector contained the oriP and Epstein-Barr nuclear antigen (EBNA)-1 elements (147), which allow for a single round of replication of the expression plasmid with each division of the cancer cell (25). The inclusion of the oriP/EBNA-1 elements within the expression plasmid enables a 10-fold increase in the level of gene expression in human U87 glioma cells (16). However, the EBNA-1 gene encodes a tumorigenic trans-acting factor (17), and this formulation may not be desirable in human gene therapy. It is possible that the EBNA-1 element would not be required if a more potent form of antisense gene therapy were used, such as DNA-based RNAi.

DNA-based RNA interference (RNAi) is a potent form of antisense gene therapy wherein an expression plasmid DNA encodes for a short hairpin RNA (shRNA) that is comprised of a stem-loop structure (6). This shRNA is processed in the cell to a RNA duplex with a 3′-overhang and this short RNA duplex mediates RNAi or post-transcriptional gene silencing. As mentioned previously, RNAi-based gene therapy offers great promise for the treatment of cancer. However, an important limiting factor is delivery of the shRNA to the cell.

In the following examples, exemplary receptor-specific nanocontainers in accordance with the present invention are prepared and studied to demonstrate the therapeutic efficacy of intravenous RNAi-based gene therapy directed at the human EGFR in mice with brain cancer. Exemplary expression plasmids are provided which lack the oriP/EBNA-1 elements and which encode for shRNA directed at specific sequences in the human EGFR mRNA. These exemplary plasmids were incorporated in HIRMAb/TfRMAb-PILs. These PILs were administered intravenously on a weekly schedule to mice with intra-cranial human brain cancer.

Examples of practice are as follows:

The materials used in the examples were obtained from commercial vendors as follows:

POPC (1-palmitoyl-2-oleoyl-sn-glycerol-3-phosphocholine), DDAB (dimethyldioctadecylammonium bromide),distearoylphosphatidylethanolamine (DSPE)-PEG²⁰⁰⁰, where PEG²⁰⁰⁰ is 2000 Dalton polyethyleneglycol, DSPE-PEG²⁰⁰⁰-maleimide (MAL), [α-³²P]dCTP (3000 Ci/mmol). N-succinimidyl[2,3-³H]propionate (³H-NSP, 101 Ci/mmol), protein G Sepharose CL-4B, 2-iminothiolane (Traut's reagent), and the bicinchoninic acid (BCA) protein assay. The anti-transferrin receptor monoclonal antibody (TfRMAb) used in this study is the 8D3 rat MAb to the mouse TfR [26]. The 8D3 MAb is specific for the mouse TfR, and is not active in human cells. The anti-insulin receptor MAb used for gene targeting to human cells is the murine 83-14 MAb to the human insulin receptor (HIR) [27]. The TfRMAb and HIRMAb were individually purified with protein G affinity chromatography from hybridoma-generated ascites. Custom olideoxynucleotides (ODN) were obtained from Biosource (Camarillo, Calif.).

### EXAMPLE 1

Design of shRNA encoding plasmid and demonstration of biological activity in cell culture. Oligodeoxynucleotide (ODN) duplexes corresponding to the various EGFR shRNAs were designed as described in the literature (28), and shown in Table 1.

TABLE 1

Design of shRNA to target EGFR mRNA.
List of ODNs used for the construction of expression plasmids.

| Plasmid | EGFR | |
|---|---|---|
| Number | mRNA (nt) | ODN sequence |
| 962 | 187-219 | Forward: |
| | | GCTGCCCCGGCCGTCCCGGAGGGTCGCATGAAGCTTGATGCGACTCTTCGG |
| | | GACGGTCGGGGTAGCGCTTTTTT |
| | | (SEQ. ID. NO. 3) |
| | | Reverse: |
| | | AATTAAAAAAGCGCTACCCCGACCGTCCCGAAGAGTCGCATCAAGCTTCAT |
| | | GCGACCCTCCGGGACGGCCGGGGCAGCGGCC |
| | | (SEQ. ID. NO. 4) |
| 963 | 2087-2119 | Forward: |
| | | GATCTTAGGCCCATTCGTTGGACAGCCTTGAAGCTTGAGGGTTGTCCGACGA |
| | | ATGGGCCTAAGATTCCTTTTTT |
| | | (SEQ. ID. NO. 5) |
| | | Reverse: |
| | | AATTAAAAAAGGAATCTTAGGCCCATTCGTCGGACAACCCTCAAGCTTCAA |
| | | GGCTGTCCAACGAATGGGCCTAAGATCGGCC |
| | | (SEQ. ID. NO. 6) |
| 964 | 3683-3715 | Forward: |
| | | GTCCTGCTGGTAGTCAGGGTTGTCCAGGCGAAGCTTGGTCTGGATAATCCTG |
| | | ACTATCAGCAGGACTTTTTTTT |
| | | (SEQ. ID. NO. 7) |
| | | Reverse: |
| | | AATTAAAAAAAAGTCCTGCTGATAGTCAGGATTATCCAGACCAAGCTTCGC |
| | | CTGGACAACCCTGACTACCAGCAGGACGGCC |
| | | (SEQ. ID. NO. 8 |
| 966 | 2346-2374 | Forward: |
| | | GTCCCTTATACACCGTGCCGAACGCACCGGAAGCTTGCGGTGCGTTCGGCG |
| | | CGGTGTGTGAGGGATTCTTTTTT |
| | | (SEQ. ID. NO. 9) |
| | | Reverse: |
| | | AATTAAAAAAGAATCCCTCACACACCGCGCCGAACGCACCGCAAGCTTCCG |
| | | GTGCGTTCGGCACGGTGTATAAGGGACGGCC |
| | | (SEQ. ID. NO. 10) |
| 967 | 2529-2557 | Forward: |
| | | GCGTGATGAGTTGCACGGTGGAGGTGAGGGAAGCTTGCTTCGCCTCCACCG |
| | | TGCAATTCATCGCGCAGTTTTTT |
| | | (SEQ. ID. NO. 11) |
| | | Reverse: |
| | | AATTAAAAAACTGCGCGATGAATTGCACGGTGGAGGCGAAGCAAGCTTCCC |
| | | TCACCTCCACCGTGCAACTCATCACGCGGCC |
| | | (SEQ. ID. NO. 12) |
| 968 | 2937-2965 | Forward: |
| | | GGATGGAGGAGATCTCGCTGGCAGGGATTGAAGCTTGAGTCTCTGCCGGCG |
| | | AGATCTCCTCCGTCCTGTTTTTT |
| | | (SEQ. ID. NO. 13) |
| | | Reverse: |
| | | AATTAAAAAACAGGACGGAGGAGATCTCGCCGGCAGAGACTCAAGCTTCA |
| | | ATCCCTGCCAGCGAGATCTCCTCCATCCGGCC |
| | | (SEQ. ID. NO. 14) |

The shRNA sequence intentionally included nucleotide mismatches in the sense strand (FIG. 1B) to reduce the formation of DNA hairpins during cloning. Because the antisense strand remains unaltered, these G-U substitutions do not interfere with the RNAi effect (29). Forward ODNs contain a U6 polymerase stop signal (T₆) (Table 1). Reverse ODNs contain 4-nucleotide overhangs specific for the EcoRI and ApaI restriction sites at 5′- and 3′-end, respectively (Table 1), to direct subcloning into the cohesive ends of a standard eukaryotic expression plasmid. The empty expression plasmid is designated clone 959 (Table 2). Complementary ODNs were heat denatured (4 min at 94° C.) and annealed at 65° C. for 16 hours in 10 mM sodium phosphate pH=7.4, 150 mM sodium chloride and 1 mM EDTA. Double stranded ODNs were ligated into the plasmid at EcoRI and ApaI sites. E. coli DH5α competent cells were transformed and clones with the correct RNAi inserts were confirmed by DNA sequencing using the T3 primer, as well as restriction endonuclease mapping with NaeI.

TABLE 2
shRNA target sites within human EGFR mRNA and biological
activity in U87 cells

| | | | thymidine |
|---|---|---|---|
| Plasmid | RNA | EGFR mRNA | incorporation |
| number | type | sequence | (% inhibition) |
| 959 | none | none | 0 |
| 962 | shRNA | 187-219 | 0 |
| 963 | shRNA | 2087-2119 | 0 |
| 966 | shRNA | 2346-2374 | 59 ± 1 |
| 967 | shRNA | 2529-2557 | 97 ± 3 |
| 968 | shRNA | 2937-2965 | 72 ± 3 |
| 964 | shRNA | 3683-3715 | 59 ± 3 |
| 882 | antisense | 2317-3006 | 100 |
| % inhibition of thymidine incorporation = [(A − B)/(A − C)] × 100, where A = thymidine incorporation with clone 959 (the empty expression plasmid), B = thymidine incorporation with clone 962, 963, 964, 966, 967, or 968, and C = thymidine incorporation with clone 882. Human U87 glioma cells were incubated with 1.0 μg plasmid DNA and 20 μg Lipofectamine in serum free medium for 4 hours. The medium was then replaced, and 24 | | | |
| # hours later [³H]-thymidine (2 μCi/mL) and 10 μM unlabeled thymidine were added and the cells were incubated for a 48 hr period prior to measurement of thymidine incorporation. Data are mean ± SE (n = 3 dishes); shRNA = short hairpin RNA, as shown in FIG. 1B. | | | |

A total of 6 anti-EGFR shRNA encoding expression plasmid DNAs were produced and designated clones 962-964 and 966-968 (Table 1). The EGFR knockdown potency of these 6 shRNA encoding expression plasmids was compared to the EGFR knockdown effect of clone 882, which is a eukaryotic expression plasmid described previously (15). Clone 882 is derived from pCEP4, is driven by the SV40 promoter, contains EBNA-1/oriP elements, and encodes for a 700 nt antisense RNA complementary to nt 2317-3006 of the human EGFR (15). The RNAi effect on the human EGFR was screened by measuring the rate of [³H]-thymidine incorporation into human U87 glioma cells in tissue culture. Forward and reverse synthetic oligodeoxynucleotides (ODNs) were designed to produce shRNAs directed at 3 broadly spaced regions of the human EGFR mRNA at nucleotides 187-219 (clone 962), 2087-2119 (clone 963), and 3683-3715 (clone 964), and the ODN sequences are given in Table 1.

The biological activity of these EGFR RNAi plasmids was tested by measuring the inhibition of [³H]-thymidine incorporation in U87 human glioma cells (Table 2). Clones 962-963 caused no knockdown of EGFR action, and the effect of clone 964 was intermediate (Table 2). Therefore, a second series of ODNs were designed to produce shRNAs directed at 3 different regions within nucleotides 2300-3000 of the human EGFR mRNA: 2346-2374 (clone 966), 2529-2557 (clone 967), and 2937-2965 (clone 968) as shown in Table 1. Whereas the knockdown of EGFR function was intermediate with clones 966 and 968, clone 967 produced a level of inhibition of [³H]-thymidine incorporation comparable to clone 882 (Table 2). The sequence and secondary structure of the shRNA produced by clone 967 is shown in FIG. 1B.

The nucleotide sequence of human EGFR mRNA is known (GENBANK ACCESSION NUMBER X00588). The nucleotide sequence begins at numbered nucleotide 1 and extends in numbered sequential positions to numbered nucleotide 5532. Based on the above results, the shRNA should be antisense to nucleotides located in the region of EGFR mRNA between numbered nucleotide positions 2346 and 3715. Preferably, the shRNA will be antisense to the region of EGFR mRNA between numbered positions 2529 and 2965. More preferably, the region of EGFR mRNA targeted with antisense is between numbered positions 2529 and 2557.

### EXAMPLE 2

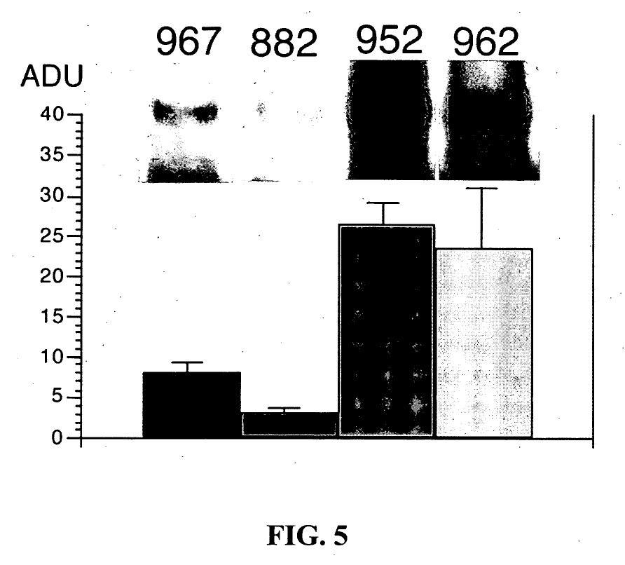

Western blotting. To confirm the inhibition of functional EGFR expression by RNAi in cell culture, we measured immunoreactive EGFR by Western blotting (FIG. 5) in cultured U87 cells following 48 hours exposure to clone 967 plasmid DNA. For controls, we measured the level of immunoreactive EGFR following exposure to clone 882 (conventional antisense gene therapy with EBNA-1), clone 962 (an ineffective anti-EGFR RNAi clone (Table 2), and clone 952 [an anti-luciferase gene RNAi clone, which should have no effect on the EGFR (ref. 19)]. Quantitation of the Western blot results show that clones 967 and 882 knocked down the EGFR 68% and 88%, respectively (FIG. 5).

Details of the Western blot are as follows:

Human U87 glioma cells were cultured on 35 mm dishes to 80% confluency. The individual plasmid DNA (clones 967, 882, 952, and 962) were applied in Lipofectamine at a dose of 1.5 mg DNA/dish for a 4 hour period. The medium was then removed and replaced with fresh medium containing 10% fetal bovine serum, and the cells were incubated at 37 C. for 48 hours. The cells were harvested in lysis buffer prior to sodium dodecyl polyacrylamide gel electrophoresis followed by blotting to nitrocellulose filters. The EGFR was detected with a commercially available antibody to the human EGFR and the Western blot signal was detected with a chemiluminescence method. The xray film was scanned into Adobe Photopshop and the integrated density was quantified by NIH Image software to give the mean and standard error results shown in FIG. 5.

### EXAMPLE 3

Demonstration of equivalency between Clones 882 (conventional antisense therapy with EBNA-1) and Clone 967 (DNA-based RNAi gene therapy without EBNA-1). U87 human glioma cells were grown in 6-well cluster dishes with MEM medium containing 10% fetal bovine serum (FBS). After the cells reached 50-60% confluence, the growth medium was replaced with 1.5 ml of serum-free MEM containing 1 μg of each plasmid DNA (clone 959, 962-964, 966-968, or 882) and 10 μl (20 μg) of Lipofectamine, and incubated for 4 hours at 37° C. The medium was replaced with MEM medium with 10% FBS and incubated for 24 hours. A final concentration of 2 μCi/ml of [³H]-thymidine and 10 μM of unlabeled thymidine were added to each dish, and dishes were incubated at 37° C. for 48 hours. The cells were harvested for measurement of [³H]-thymidine incorporation as described previously (14). The transfection of the U87 cells with Lipofectamine demonstrated that clone 967 was the most potent clone causing RNA interference of EGFR expression, and at high doses was just as effective as clone 882 (Table 2).

To further examine the relative potentcy of clone 882 and 967, a dose response study with clone 967 was performed, in parallel with a dose response study for clone 882, which encodes for the 700 nt EGFR antisense RNA (14). In these dose response studies, the clone 882 or clone 967 DNA was delivered to human glioma cells in cell culture with the HIRMAb-targeted PIL. U87 cells were grown on 35-mm collagen-treated dishes. After the cells reached 50-60% confluence, the medium was aspirated and 2 ml of fresh MEM medium with 10% FBS and HIRMAb-PILs encapsulated with clone 967 or clone 882 at a dose of 1.4, 0.14, 0.014 or 0.0014 μg DNA/dish were added. The cells were incubated for 5 days at 37° C. During this period, 2 ml fresh medium was added after 3 days of incubation. At 5 days, the medium was aspirated, and 2 ml of fresh growth medium containing 2 μCi/ml of [³H]-thymidine and 10 μM of unlabeled thymidine were added to each dish, followed by a 48-hour incubation at 37° C. At the end of the incubation, [³H]-thymidine incorporation was measured and expressed as nmol thymidine incorporated per mg cell protein, as described previously (14). Clone 967 or 882 plasmid DNA were then encapsulated in HIRMAb-targeted PILs and added to U87 cells without Lipofectamine at varying doses of plasmid DNA ranging from 1.4-1400 ng/dish. Either plasmid DNA was equally active in suppressing thymidine incorporation with an ED₅₀ of approximately 100 ng/dish (FIG. 1C). This tissue culture experiment showed it was possible to produce a RNAi expression plasmid against the human EGFR that lacked the EBNA-1 gene (clone 967), but which was equally effective with a plasmid encoding a long 700 nt RNA under the influence of EBNA-1 (clone 882). Before this study, however, the relative efficacy of clone 967 in vivo was not known, nor was it known whether clone 967 could prolong survival time in animals with human brain cancer.

Synthesis of pegylated immunoliposomes. Clone 967 or 882 plasmid DNA was encapsulated in PILs as described previously (U.S. Pat. No. 6,372,250 and (14)). The liposome was 85-100 nm in diameter and the surface of the liposome was conjugated with several thousand strands of 2000 Da polyethyleneglycol (PEG). The tips of about 1-2% of the PEG strands were conjugated with 83-14 HIRMAb and the 8D3 TfRMAb, as described previously (15). Any plasmid DNA not encapsulated in the interior of the liposome was quantitatively removed by exhaustive nuclease treatment (23). In a typical synthesis, 30-40% of the initial plasmid DNA (200 μg) was encapsulated within 20 μmol of lipid, and each liposome had a range of 43-87 MAb molecules conjugated to the PEG strands (14).

### EXAMPLE 4

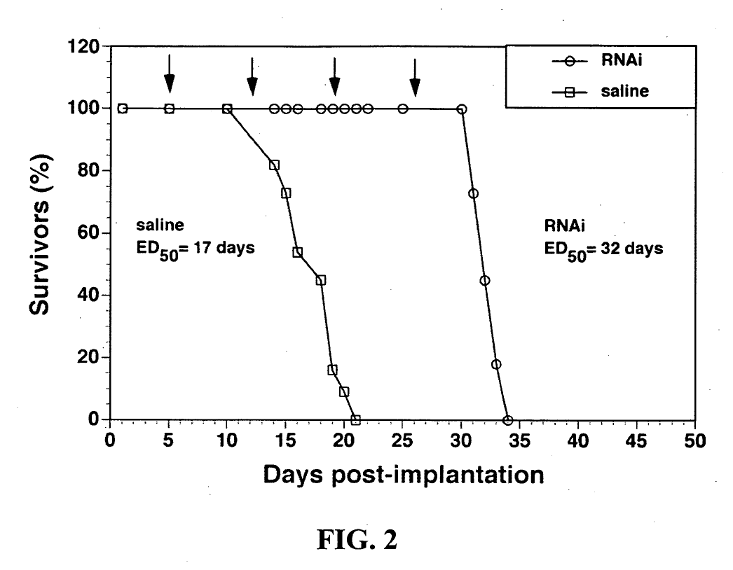

Increase in survival with intravenous RNAi gene therapy of intra-cranial brain cancer. Female severe combined immunodeficient (scid) mice weighing 19-21 g were purchased from the Jackson Laboratory (Bar Harbor, Me.). A burr hole was drilled 2.5 mm to the right of midline and 1 mm anterior to bregma. U87 glioma cells were suspended in serum-free MEM containing 1.2% methylcellulose. Five μl of cell suspension (5×10⁵ cells) were injected into the right caudate-putamen nucleus at a depth of 3.5 mm over 2 min, using a 10-μl Hamilton syringe with fixed needle. The animals were treated intravenously once a week starting at day 5 after implantation. By 5 days after the implantation of 500,000 U87 cells, the tumor is large and fills the entire volume of the striatum in brain (32). Weekly intravenous gene therapy was administered at 5, 12, 19 and 26 days after implantation. Mice were treated with either saline or 5 μg/mouse of clone 967 DNA encapsulated in the HIRMAb/mTfRMAb-PILs. Human U87 glioma cells were implanted in the caudate-putamen nucleus of adult scid mice, which causes death at 14-20 days secondary to the growth of large intracranial tumors. Starting on day 5 post-implantation, mice were treated with weekly intravenous injections of either saline or 5 μg/mouse of clone 967 plasmid DNA encapsulated in PILs that were doubly targeted with both the 83-14 murine MAb to the HIR and the 8D3 rat MAb to the mouse TfR (FIG. 1A). The saline treated mice died between 14 and 20 days post-implantation with an ED₅₀ of 17 days (FIG. 2). The mice treated with intravenous RNAi gene therapy died between 31 and 34 days post-implanation with an ED₅₀ of 32 days (FIG. 2).

### EXAMPLE 5

Reduction of EGFR in brain tumors in vivo with RNAi gene therapy and PIL gene targeting. Brains were removed immediately after sacrifice, and cut into coronal slabs from the center of tumor. Slabs were embedded in O.C.T. medium, and frozen in dry ice powder. Frozen sections (20 μm) of mouse brain were cut on a Mikron HM505E cryostat. Sections were fixed in cold 100% methanol for 20 min at −20° C. For confocal microscopy, nonspecific binding of proteins was blocked with 10% donkey serum-phosphate-buffered saline (PBS) for 30 min. The sections were incubated in primary antibody overnight at 4° C. The primary antibodies were the rat 8D3 MAb to the mouse TfR (10 μg/ml), and the mouse 528 MAb against the human EGFR (10 μg/ml). After a PBS wash, a rhodamine-conjugated donkey anti-rat IgG secondary antibody, 5 μg/ml, was added for 30 min at room temperature. The slides were then washed and incubated with fluorescein-conjugated goat anti-mouse IgG at 5 μg/ml for 30 min at room temperature. The sections were mounted on slides, and viewed with a 40X objective and a Zeiss LSM 5 PASCAL confocal microscope with dual argon and helium/neon lasers. The sample was scanned in multitrack mode to avoid leakage of the fluorescein signal into the rhodamine channel. Sections were scanned at intervals of 0.8 μm and reconstructed with Zeiss LSM software. Control experiments used either a rat IgG (Sigma) or a mouse IgG1 (Sigma) as primary antibodies in lieu of the rat anti-mouse TfR or the mouse anti-human EGFR antibody, respectively.

Immunocytochemistry was performed by the avidin-biotin complex. (ABC) immunoperoxidase method (Vector Laboratories). To stain the human EGFR, the mouse 528 MAb anti-human EGFR was used as the primary antibody (33); to stain the mouse TfR, the rat 8D3 MAb anti-mouse TfR was used as the primary antibody (15). Endogenous peroxidase was blocked with 0.3% H₂O₂ in 0.3% horse serum-phosphate-buffered saline (PBS) for 30 min; nonspecific binding of proteins was blocked with 3% horse or rabbit serum in PBS for 30 min. For mouse TfR staining using rat 8D3 MAb, rabbit serum was used in the blocking steps. Sections were then incubated in 10 μg/ml of primary antibody overnight at 4 C. Identical concentrations of isotype control antibody were also used as primary antibody. Mouse IgG1 was used as the isotype antibody for 528 MAb, and rat IgG was used as the isotype control antibody for 8D3 MAb. After incubation and wash in PBS, sections were incubated in either biotinylated horse anti-mouse IgG (for 528 MAb) or biotinylated rabbit anti-mouse IgG (for 8D3 MAb) for 30 min, prior to color development with AEC. Slides were not counter-stained.

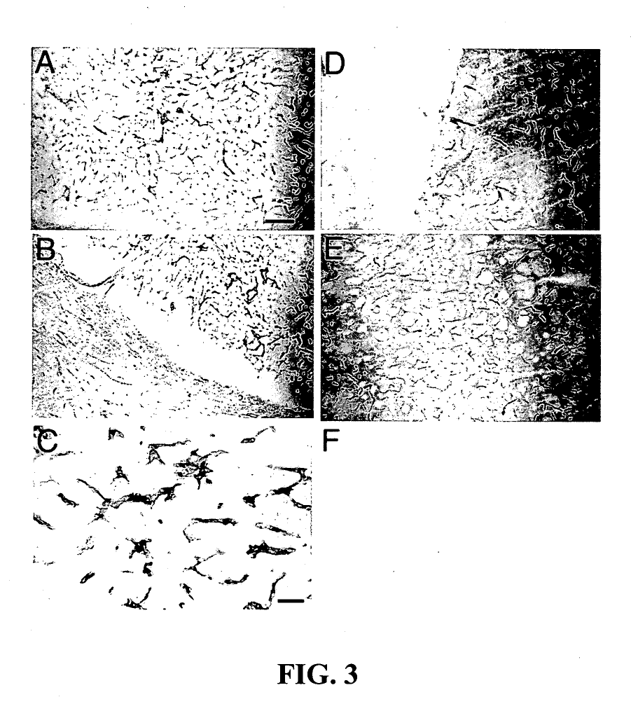

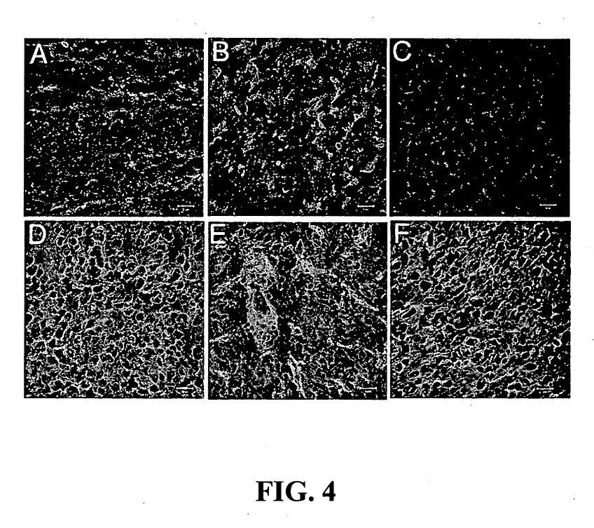

The tumors were examined at autopsy by immunocytochemistry using the rat 8D3 MAb to the mouse TfR (FIG. 3). The tumors from the saline treated animals were well vascularized and expressed the murine TfR (FIGS. 3A, B, C). FIG. 3B shows the immunoreactive murine TfR on the vascular endothelium of normal brain and the tumor. A blood vessel originating from normal brain and extending into the tumor is visible (FIG. 3B). The border between the tumor and the normal brain frequently had a low vascular density as shown in FIG. 3B. The vascular density in the tumors of the RNAi treated mice was generally low as shown in FIG. 3D, although EGFR RNAi gene therapy did not cause a decrease in vascular density in normal brain as shown in FIG. 3E. Confocal microscopy of the tumor sections following double immune labeling with both the rat 8D3 MAb to the vascular mouse TfR (red channel) and the murine 83-14 MAb to the tumor HIR (green channel) is shown in FIG. 4. There is down-regulation of the immunoreactive EGFR in the RNAi treated tumors (FIGS. 4A, B, and C) relative to the saline treated tumors (FIGS. 4D, E, and F).

### EXAMPLE 6

RNAi of cancer specific mutants of the EGFR. Many solid cancers, including brain cancer, specifically express mutant forms of the EGFR (34, 35). The most common mutant is the EGFRvIII mutant (34, 35), which is up-regulated independent of ligand binding. The EGFRvIII mutant has a specific nucleotide sequence that is not present in the wild type EGFR or any other gene. Therefore, a plasmid expressing an shRNA directed against the unique EGFRvIII mutant would be 100% specific for cancer, and would not suppress the EGFR in non-cancer cells. ODN duplexes corresponding to the various shRNAs directed to the splice site of the hEGFR vIII were designed similar to that described in Example 1, and the EGFRvIII specific shRNAs are shown in Table 4.

TABLE 4

Design of shRNA to target EGFRvIII mRNA.
List of ODNs used for the construction of expression plasmids.

| Plasmid | EGFR vIII | |
|---|---|---|
| Number | mRNA (nt) | ODN sequence |
| vIII-1 | 242-269 | Forward: |
| | | TCACCACATAATTACCTTTCTTTTCCTCGAAGCTTGGAGGGAAAGGAAGGTA |
| | | GTTGTGTGGTGATCTTTTTT |
| | | (SEQ. ID NO. 15) |
| | | Reverse: |
| | | AATTAAAAAAGATCACCACACAACTACCTTCCTTTCCCTCCAAGCTTCGAGG |
| | | AAAAGAAAGGTAATTATGTGGTGAGGCC |
| | | (SEQ, ID. NO 16) |
| vIII-2 | 244-271 | Forward: |
| | | TGTCACCACATAATTACCTTTCTTTTCCGAAGCTTGGGGAAAGGAAGGTAGT |
| | | TGTGTGGTGACATCTTTTTT |
| | | (SEQ. ID NO. 17) |
| | | Reverse: |
| | | AATTAAAAAAGATGTCACCACACAACTACCTTCCTTTCCCCAAGCTTCGGAA |
| | | AAGAAAGGTAATTATGTGGTGACAGGCC |
| | | (SEQ. ID. NO. 18) |
| vIII-3 | 245-272 | Forward: |
| | | CTGTCACCACATAATTACCTTTCTTTTCGAAGCTTGGGAAAGGAAGGTAGTT |
| | | GTGTGGTGACAGTCTTTTTT |
| | | (SEQ, ID. NO. 19) |
| | | Reverse: |
| | | AATTAAAAAAGACTGTCACCACACAACTACCTTCCTTTCCCAAGCTTCGAAA |
| | | AGAAAGGTAATTATGTGGTGACAGGGCC |
| | | (SEQ. ID. NO. 20) |

Nucleotide overhangs to the EcoRI and ApaI restriction sites at 5′- and 3′-end of reverse ODNs are underlined. The EGFRvIII is a mutant form of the EGFR that is expressed only in cancer.

The shRNA sequence intentionally included nucleotide mismatches in the sense strand to reduce the formation of DNA hairpins during cloning. Because the antisense strand remains unaltered, these G-U substitutions do not interfere with the RNAi effect. Forward ODNs contain a U6 polymerase stop signal (T₆) (Table 3). Reverse ODNs contain 4-nucleotide overhangs specific for the EcoRI and ApaI restriction sites at 5′- and 3′-end, respectively, to direct subcloning into the cohesive ends of the U6 expression vector. Using methods taught in this invention, those skilled in the art can use the ODNs described in Table 3 to produce expression plasmids that enable DNA-based RNAi gene therapy of cancers in either brain or other tissues that are oncogenic on the basis of the unique expression of the EGFRvIII mutant. This form of cancer gene therapy is highly desirable, since the EGFR in normal cells would not be affected by the shRNA that selectively targets the unique sequence within the EGFRvIII mRNA.

The above examples demonstrate that it is possible to knockdown EGFR gene expression with RNAi-based gene therapy that employs expression plasmids encoding a shRNA directed at nucleotides 2529-2557 of the human EGFR mRNA (Table 2). Also, EGFR expression knockdown is demonstrated by the inhibition of thymidine incorporation in human U87 glioma cells in tissue culture (Table 2), and in vivo by the decrease in brain cancer expression of immunoreactive EGFR (FIG. 4). Finally, weekly intravenous EGFR RNAi gene therapy resulted in an 88% increase in survival time (FIG. 2), despite delaying treatment until 5 days after implantation when the tumor size is large (32).

The discovery of RNAi-active target sequences within the human EGFR transcript required several iterations (Table 1 and Table 2). These findings were consistent with the suggestion of McManus and Sharp (6), that approximately 1 out of 5 target sequences yield therapeutic effects in RNAi. Prior work had shown that EGFR gene expression could be inhibited with RNA duplexes delivered to cultured cells with oligofectamine and without the use of DNA vectors encoding shRNAs (18). The present examples demonstrate that EGFR gene expression can be inhibited with RNAi-based expression plasmids that produce an intracellular shRNA, and that the DNA-based RNAi is effective both in cell culture and in vivo in human cells. On the basis of the cell culture work evaluating thymidine incorporation, clone 967 was chosen for further evaluation of RNAi-based gene therapy to knock down human EGFR gene expression. Clone 967 produces a shRNA directed against nucleotides 2529-2557 (FIG. 1B), and this target sequence is within the 700 nucleotide region of the human EGFR mRNA that is targeted by antisense RNA expressed by clone 882 (15). Clone 967 and clone 882 equally inhibit thymidine incorporation in human U87 cells (FIG. 1C), and this is evidence for the increased potency of RNAi-based forms of antisense gene therapy. The clone 882 plasmid contains the EBNA-1/oriP gene element (14), which enables a 10-fold increase in expression of the trans-gene in cultured U87 cells (16). Therefore, the increased potency of the RNAi approach to antisense gene therapy enabled the elimination of the potentially tumorigenic EBNA-1 element in the expression plasmid.

Clone 967 was delivered to cultured U87 cells with HIRMAb-targeted PILs, and clone 967 knocked down EGFR function in a dose dependent mechanism, with respect to inhibition of thymidine incorporation (FIG. 1C) with an ED₅₀ of approximately 100 ng plasmid DNA/dish. The expression of immunoreactive EGFR in the brain cancer is still markedly diminished at 5-8 days following the last intravenous dose of EGFR RNAi gene therapy (FIG. 4).

The above examples show an 88% increase in survival time with weekly intravenous gene therapy using clone 967 encapsulated in HIRMAb/TfRMAb-PILs (FIG. 2). This increase in survival time with weekly intravenous gene therapy is comparable to the prolongation of survival time in mice treated with high daily doses of the EGFR-tyrosine kinase inhibitor, ZD1839 (Iressa) (11). Daily oral Iressa chemotherapy was initiated when the tumor was macroscopically visible at 3 days following the intracranial implantation of 100,000 glioma cells (11). However, Iressa was not effective in the treatment of brain cancer expressing mutant forms of the EGFR (11). Many primary and metastatic brain cancers express mutations of the human EGFR (34-35), and it is possible to design RNAi-based gene therapy that will knock down both wild type and mutant EGFR, as described in Example 5.

In summary, the examples of the invention demonstrate that weekly intravenous RNAi gene therapy directed against the human EGFR causes an 88% increase in survival time in adult mice with intra-cranial human brain cancer. The PIL non-viral gene transfer technology can be used to both knock down tumorigenic genes and to replace mutated tumor suppressor genes in brain cancer. The efficacy of the PIL non-viral gene transfer technology has been demonstrated in primates, and levels of gene expression in primate brain are 50-fold greater than comparable levels of gene expression in rodent brain (36). PILs carrying therapeutic genes can be delivered to human brain cancer using genetically engineered monoclonal antibodies. A chimeric HIRMAb (37) has the same activity in terms of binding to the human BBB in vitro, or transport across the primate BBB in vivo, as the original murine HIRMAb used in these examples. The high therapeutic efficacy of the PIL gene transfer technology is possible because this approach delivers therapeutic genes to brain and other organs via the transvascular route (22).

The above examples show that the receptor-mediated nanocontainers of the present invention are effective in treating human brain cancer in a mouse model. For human use, it would be necessary to only use 1 targeting ligand, the HIRMAb, in the formulation of the PIL (FIG. 1A). This is because the HIR is expressed on both the tumor cell membrane and the tumor capillary of human brain cancer (38). Clone 967 produces a shRNA against the human EGFR. The murine HIRMAb used in these studies to formulate the PIL could not be used in humans. However, genetically engineered forms of the HIRMAb have been produced and are now available for use in humans ((37) and U.S. patent application Ser. No. 10/307,276).

Having thus described exemplary embodiments of the present invention, it should be noted by those skilled in the art that the within disclosures are exemplary only and that various other alternatives, adaptations and modifications may be made within the scope of the present invention. Accordingly, the present invention is not limited to the above preferred embodiments and examples, but is only limited by the following claims.

### BIBLIOGRAPHY

1. Ram, Z. et al. Therapy of malignant brain tumors by intratumoral implantation of retroviral vector-producing cells. Nat. Med. 3, 1354-1361 (1997).
2. Driesse, M. J., et al. Intracerebral injection of adenovirus harboring the HSVtk gene combined with ganciclovir administration: toxicity study in non-human primates. Gene Ther. 5, 1122-1129 (1998).
3. Dewey, R. A., et al. Chronic brain inflammation and persistent herpes simplex virus 1 thymidine kinase expression in survivors of syngeneic glioma treated by adenovirus-mediated gene therapy: implications for clinical trials. Nat. Med. 5, 1256-1263.
4. Twombly, R. For gene therapy, now-quantified risks are deemed troubling. J. Natl Cancer Inst. 95, 1032-1033 (2003).
5. Nakai, H., Montini, E., Fues, S., Storm T. A., Grompe, M., Kay, M. A. AAV serotype 2 vectors preferentially integrate into active genes in mice. Nal. Genet. 34, 297-302 (2003).
6. McManus, M. T. & Sharp, P. A. Gene silencing in mammals by small interfering RNAs. Nat. Rev. Genet. 3, 737-747 (2002).
7. Elbashir, S. M., Harborth, J., Weber, K., and Tuschl, T. Analysis of gene function in somatic mammalian cells using small interfering RNAs. Methods 26, 199-213 (2002).
8. Verma, I. M., and Somia, N. Gene therapy—promises, problems and prospects. Nature 389, 239-242 (1997).
9. Kuan, C. T., Wikstrand, C. J. & Bigner, D. D. EGF mutant receptor vIII as a molecular target in cancer therapy. Endocr. Relat. Cancer 8, 83-96 (2001).
10. Nicholson, R. I., Gee, J. M., and Harper, M. E. EGFR and cancer prognosis. Eur. J. Cancer 37 Suppl 4, S9-15 (2001).
11. Heimberger, A. B. et al. Brain tumors in mice are susceptible to blockade of epidermal growth factor receptor (EGFR) with the oral, specific, EGFR-tyrosine kinase inhibitor ZD1839 (iressa). Clin. Cancer Res. 8, 3496-3502 (2002).
12. Mishima, K., et al. Growth suppression of intracranial xenografted glioblastomas overexpressing mutant epidermal growth factor receptors by systemic administration of monoclonal antibody (mAb) 806, a novel monoclonal antibody directed to the receptor. Cancer Res. 61, 5349-5354 (2001).
13. Moroni, M. C., Willingham, M. C., and Beguinot, L. EGF-R antisense RNA blocks expression of the epidermal growth factor receptor and suprresses the transforming phenotype of a human carcinoma cell line. J. Biol. Chem. 267, 2714-2722 (1992).
14. Zhang, Y., Lee, H. J., Boado, R. J., & Pardridge, W. M. Receptor-mediated delivery of an antisense gene to human brain cancer cells. J Gene Med. 4, 183-194 (2002).
15. Zhang, Y., Zhu, C. & Pardridge, W. M. Antisense gene therapy of brain cancer with an artificial virus gene delivery system. Mol. Ther. 6, 67-72 (2002).
16. Zhang, Y., Boado, R. J. & Pardridge, W. M. Marked enhancement in gene expression by targeting the human insulin receptor. J Gene Med. 5, 157-163 (2003).
17. Snudden, D. K., Smith, P. R., Lai, D., Ng, M. H. & Griffin, B. E. Alterations in the structure of the EBV nuclear antigen, EBNA1, in epithelial cell tumours. Oncogene 10, 1545-1552 (1995).
18. Nagy, P., Arndt-Jovin, D. J. & Jovin, T. M. Small interfering RNAs suppress the expression of endogenous and GFP-fused epidermal growth factor receptor (erbB1) and induce apoptosis in erbB1-overexpressing cells. Exp Cell Res 285, 39-49 (2003).
19. Zhang, Y., Boado, R. J. & Pardridge, W. M. In vivo knock-down of gene expression in brain cancer with intravenous RNAi in adult rats. J Gene Med., 5, 1039-1045 (2003).
20. Wong, A. J., et al. Increased expression of the epidermal growth factor receptor gene in malignant gliomas is invariably associated with gene amplification. Proc. Natl. Acad. Sci. USA 84, 6899-6903 (1987).
21. Libermann, T. A., Nusbaum, H. R., Razon, N., et al. Amplification, enhanced expression and possible rearrangement of EGF receptor gene in primary human brain tumors of glial origin. Nature 313, 144-147 (1985).
22. Pardridge, W. M. Drug and gene delivery to the brain: the vascular route. Neuron 36, 555-558 (2002).
23. Shi, N. & Pardridge, W. M. Non-invasive gene targeting to the brain. Proc. Natl. Acad. Sci., U.S.A. 97, 7567-7572 (2000).
24. Pardridge, W. M. Drug and gene targeting to the brain with molecular Trojan horses. Nature Reviews-Drug Discovery 1, 131-139 (2002).
25. Makrides, S. C. Components of vectors for gene transfer and expression in mammalian cells. Protein Expr. Purif 17, 183-202 (1999).
26. Lee, H. J., Engelhardt, B., Lesley, J., Bickel, U. & Pardridge, W. M. (2000). Targeting rat anti-mouse transferrin receptor monoclonal antibodies through the blood-brain barrier in the mouse. J. Pharmacol. Exp. Ther. 292, 1048-1052.
27. Pardridge, W. M., Kang, Y.-S., Buciak, J. L. & Yang, J. (1995). Human insulin receptor monoclonal antibody undergoes high affinity binding to human brain capillaries in vitro and rapid transcytosis through the blood-brain barrier in vivo in the primate. Pharm. Res. 12, 807-816.
28. Paddison, P., Caudy, A., Bernstein, E., Hannon, G., & Conklin, D. Short hairpin RNAs (shRNAs) induce sequence-specific silencing in mammalian cells. Genes Dev. 16, 948-958 (2002).
29. Yu, J. Y., Taylor, J., DeRuiter, S. L., Vojtek, A. B. & Turner, D. L. Simultaneous inhibition of GSK3alphna and GSK3beta using hairpin siRNA expression vectors. Mol. Ther. 7, 228-236 (2003).
30. Hernandez, M., Barrero, M. J., Crespo, M. S. & Nieto, M. L. (2000). Lysophosphatidic acid inhibits Ca²⁺ signaling in response to epidermal growth factor receptor stimulation in human astrocytoma cells by a mechanism involving phospholipase C and a Gᵢ protein. J. Neurochem. 75, 1575-1582.
31. Stout, C. E., Costantin, J. L., Naus, C. C., & Charles, A. C. (2002). Intercellular calcium signaling in astrocytes via ATP release through connexin hemichannels. J. Biol. Chem. 277, 10482-10488.
32. Lal, S. et al. An implantable guide-screw system for brain tumor studies in small animals. J. Neurosurg. 92, 326-333 (2000).
33. Kurihara, A. & Pardridge, W. M. Imaging brain tumors by targeting peptide radiopharmaceuticals through the blood-brain barrier. Canc. Res. 54, 6159-6163 (1999).
34. Luwor, R. B. et al. Monoclonal antibody 806 inhibits the growth of tumor xenografts expressing either the de2-7 or amplified epidermal growth factor (EGFR) but not wild-type EGFR. Cancer Res. 61, 3496-3502 (2002).
35. Lal, A. et al. Mutant epidermal growth factor receptor up-regulates molecular effectors of tumor invasion. Cancer Res. 62, 5355-5361 (2001).
36. Zhang, Y., Schlachetzki, F. & Pardridge, W. M. Global non-viral gene transfer to the primate brain following intravenous administration. Mol. Ther. 7, 11-18 (2003).
37. Coloma, M. J. et al. Transport across the primate blood-brain barrier of a genetically engineered chimeric monoclonal antibody to the human insulin receptor. Pharm. Res. 17, 266-274 (2000).
38. Pardridge, W. M. Brain Drug Targeting: The Future of Brain Drug Development. Cambridge University Press, Cambridge, United Kingdom, pp 1-370 (2001).
39. Traxler, P. et al. Tyrosine kinase inhibitors: from rational design to clinical trials. Medicinal Research Reviews, 21, 499-512 (2001).
40. Clark, A. F. and Yorio, T. Ophthalmic drug discovery. Nature Reviews Drug Discovery, 2, 448-459 (2003).

1. A receptor-specific nanocontainer for delivering a gene encoding short hairpin RNA to a cell having a receptor, said receptor-specific nanocontainer comprising:
a liposome having an exterior surface and an internal compartment;
a gene comprising a sufficient amount of genetic information to encode a short hairpin RNA, said gene being located within the internal compartment of said liposome;
a plurality of receptor targeting agents which are capable of targeting said receptor; and
a plurality of conjugation agents wherein each targeting agent is connected to the exterior surface of said liposome via at least one of said conjugation agents.

2. The receptor-specific nanocontainer for delivering a gene encoding short hairpin RNA to a cell having a receptor according to claim 1 wherein said short hairpin RNA comprises a nucleotide sequence that is antisense to at least a portion of mRNA selected from the group consisting of mRNAs encoding the human epidermal growth factor receptor, mutants of the EGFR, HER2, HER3, HER4, fibroblast growth factor receptor (FGFR), platelet derived growth factor receptor (PDGFR), insulin-like growth factor receptor-1 (IGFR1), transforming growth factor-α (TGF-α), vascular endothelial growth factor (VEGF) or its receptor, VEGFR, altered protein kinases including the Bcr-Abl, c-Met, c-Kit, ras, raf, or CdKs.

3. The receptor-specific nanocontainer for delivering a gene encoding short hairpin RNA to a cell having a receptor according to claim 2 wherein said short hairpin RNA comprises a nucleotide sequence that is antisense to a portion of human epidermal growth factor receptor mRNA, said human epidermal growth factor receptor mRNA comprising a nucleotide sequence having numbered nucleotides from 1 to 5532.

4. The receptor-specific nanocontainer for delivering a gene encoding short hairpin RNA to a cell having a receptor according to claim 3 wherein said short hairpin RNA comprises a nucleotide sequence that is antisense to a portion of said human epidermal growth factor receptor mRNA that is located between numbered nucleotides 2300 and 3800.

5. The receptor-specific nanocontainer for delivering a gene encoding short hairpin RNA to a cell having a receptor according to claim 4 wherein said portion of said human epidermal growth factor receptor mRNA is located between numbered nucleotides 2500 and 3000

6. The receptor-specific nanocontainer for delivering a gene encoding short hairpin RNA to a cell having a receptor according to claim 5 wherein said portion of said human epidermal growth factor receptor mRNA is located between number nucleotides 2500 and 2600.

7. The receptor-specific nanocontainer for delivering a gene encoding short hairpin RNA to a cell having a receptor according to claim 1 wherein said liposome exterior surface defines a sphere having a diameter of less than 200 nanometers.

8. The receptor-specific nanocontainer for delivering a gene encoding short hairpin RNA to a cell having a receptor according to claim 1 wherein between 5 and 500 receptor-targeting agents are conjugated to the exterior surface of said liposome.

9. The receptor-specific nanocontainer for delivering a gene encoding short hairpin RNA to a cell having a receptor according to claim 1 wherein said conjugation agent is selected from the group consisting of polyetheylene glycol, sphingomyelin and organic polymers.

10. The receptor-specific nanocontainer for delivering a gene encoding short hairpin RNA to a cell having a receptor according to claim 9 wherein the molecular weight of said conjugation agent is between 1000 and 50,000 Daltons.

11. The receptor-specific nanocontainer for delivering a gene encoding short hairpin RNA to a cell having a receptor according to claim 1 wherein from 100 to 10,000 conjugation agents are attached to the exterior surface of said liposome.

12. The receptor-specific nanocontainer for delivering a gene encoding short hairpin RNA to a cell having a receptor according to claim 1 wherein said targeting agents are capable of targeting a receptor located on a solid tumor.

13. The receptor-specific nanocontainer for delivering a gene encoding short hairpin RNA to a cell having a receptor according to claim 12 wherein said solid tumor is selected from the group consisting of brain tumors, liver tumors, lung tumors, spleen tumors, breast tumors, kidney tumors, prostate tumors, ovary tumors, eye tumors, gastrointestinal tumors, bone tumors, blood tumors, endocrine tumors, skin tumors, or lymph node tumors.

14. The receptor-specific nanocontainer for delivering a gene encoding short hairpin RNA to a cell having a receptor according to claim 13 wherein said solid tumor is a brain tumor.

15. The receptor-specific nanocontainer for delivering a gene encoding short hairpin RNA to a cell having a receptor according to claim 1 wherein said targeting agent is capable of targeting a receptor selected from the group consisting of insulin receptor, transferrin receptor, insulin-like growth factor receptor, leptin receptor, low density lipoprotein receptor fibroblast growth factor receptor.

16. A composition comprising the receptor-specific nanocontainer according to claim 1 and a pharmaceutically acceptable carrier for said receptor-specific nanocontainer.

17. A composition comprising the receptor-specific nanocontainer according to claim 16 wherein said cell to which said gene encoding said short hairpin RNA is to be delivered is located within an animal.

18. A method for delivering a short hairpin RNA to a cell having a receptor, said method comprising the step of administering to an animal an effective amount of a preparation comprising:
a) a receptor-specific nanocontainer comprising:
a liposome having an exterior surface and an internal compartment;
a gene comprising a sufficient amount of genetic information to encode a short hairpin RNA, said gene being located within the internal compartment of said liposome;
a plurality of receptor targeting agents which are capable of targeting said receptor; and
a plurality of conjugation agents wherein each targeting agent is connected to the exterior surface of said liposome via at least one of said conjugation agents; and
b) a pharmaceutically acceptable carrier for said receptor-specific nanocontainer.

19. The method for delivering a gene encoding short hairpin RNA to a cell having a receptor according to claim 18 wherein said short hairpin RNA comprises a nucleotide sequence that is antisense to at least a portion of mRNAs encoding the human epidermal growth factor receptor, mutants of the EGFR, HER2, HER3, HER4, fibroblast growth factor receptor (FGFR), platelet derived growth factor receptor (PDGFR), insulin-like growth factor receptor-1 (IGFR1) transforming growth factor-α (TGF-α), vascular endothelial growth factor (VEGF) or its receptor, VEGFR, altered protein kinases including the Bcr-Abl, c-Met, c-Kit, ras, raf, or CdK.s

20. The method for delivering a gene encoding short hairpin RNA to a cell having a receptor according to claim 19 wherein said short hairpin RNA comprises a nucleotide sequence that is antisense to a portion of human epidermal growth factor receptor mRNA, said human epidermal growth factor receptor mRNA comprising a nucleotide sequence having numbered nucleotides from 1 to 5532.

21. The method for delivering a gene encoding short hairpin RNA to a cell having a receptor according to claim 20 wherein said short hairpin RNA comprises a nucleotide sequence that is antisense to a portion of said human epidermal growth factor receptor mRNA that is located between numbered nucleotides 2300 and 3800.

22. The method for delivering a gene encoding short hairpin RNA to a cell having a receptor according to claim 21 wherein said portion of said human epidermal growth factor receptor mRNA is located between numbered nucleotides 2500 and 3000.

23. The method for delivering a gene encoding short hairpin RNA to a cell having a receptor according to claim 22 wherein said portion of said human epidermal growth factor receptor mRNA is located between number nucleotides 2500 and 2600.

24. The method for delivering a gene encoding short hairpin RNA to a cell having a receptor according to claim 18 wherein said liposome exterior surface defines a sphere having a diameter of less than 200 nanometers.

25. The method for delivering a gene encoding short hairpin RNA to a cell having a receptor according to claim 18 wherein 5 and 500 receptor-targeting agents are conjugated to the exterior surface of said liposome.

26. The method for delivering a gene encoding short hairpin RNA to a cell having a receptor according to claim 18 wherein said conjugation agent is selected from the group consisting of polyetheylene glycol, sphingomyelin and organic polymers.

27. The method for delivering a gene encoding short hairpin RNA to a cell having a receptor according to claim 26 wherein the molecular weight of said conjugation agent is between 1000 and 50,000 Daltons.

28. The method for delivering a gene encoding short hairpin RNA to a cell having a receptor according to claim 18 wherein from 100 to 10,000 conjugation agents are attached to the exterior surface of said liposome.

29. The method for delivering a gene encoding short hairpin RNA to a cell having a receptor according to claim 18 wherein said targeting agents are capable of targeting a receptor located on a solid tumor.

30. The method for delivering a gene encoding short hairpin RNA to a cell having a receptor according to claim 18 wherein said solid tumor is selected from the group consisting of brain tumors, liver tumors, lung tumors, spleen tumors, breast tumors, kidney tumors, prostate tumors, ovary tumors, eye tumors, gastrointestinal tumors, bone tumors, blood tumors, endocrine tumors, skin tumors, or lymph node tumors.

31. The method for delivering a gene encoding short hairpin RNA to a cell having a receptor according to claim 18 wherein said solid tumor is a brain tumor.

32. The method for delivering a gene encoding short hairpin RNA to a cell having a receptor according to claim 18 wherein said targeting agent is capable of targeting a receptor selected from the group consisting of insulin receptor, transferrin receptor, insulin-like growth factor receptor, leptin receptor, low density lipoprotein receptor, fibroblast growth factor.


SEQUENCE LISTING (20 sequences)

SEQ ID NO: 1 - DNA (nucleic acid), 29 bp; single-stranded, linear
ccucaccuccaccgugcaacucaucacgc

SEQ ID NO: 2 - DNA (nucleic acid), 74 bp; single-stranded, linear
gcgugaugaguugcacgguggaggugagggaagcuugcuucgccuccaccgugcaauucaucgcgcaguuuuuu

SEQ ID NO: 3 - DNA (nucleic acid), 74 bp; single-stranded, linear
gctgccccggccgtcccggagggtcgcatgaagcttgatgcgactcttcgggacggtcggggtagcgctttttt

SEQ ID NO: 4 - DNA (nucleic acid), 82 bp; single-stranded, linear
aattaaaaaagcgctaccccgaccgtcccgaagagtcgcatcaagcttcatgcgaccctccgggacggccggggcagcggcc

SEQ ID NO: 5 - DNA (nucleic acid), 74 bp; single-stranded, linear
gatcttaggcccattcgttggacagccttgaagcttgagggttgtccgacgaatgggcctaagattcctttttt

SEQ ID NO: 6 - DNA (nucleic acid), 82 bp; single-stranded, linear
aattaaaaaaggaatcttaggcccattcgtcggacaaccctcaagcttcaaggctgtccaacgaatgggcctaagatcggcc

SEQ ID NO: 7 - DNA (nucleic acid), 74 bp; single-stranded, linear
gtcctgctggtagtcagggttgtccaggcgaagcttggtctggataatcctgactatcagcaggactttttttt

SEQ ID NO: 8 - DNA (nucleic acid), 82 bp; single-stranded, linear
aattaaaaaaaagtcctgctgatagtcaggattatccagaccaagcttcgcctggacaaccctgactaccagcaggacggcc

SEQ ID NO: 9 - DNA (nucleic acid), 74 bp; single-stranded, linear
gtcccttatacaccgtgccgaacgcaccggaagcttgcggtgcgttcggcgcggtgtgtgagggattctttttt

SEQ ID NO: 10 - DNA (nucleic acid), 82 bp; single-stranded, linear
aattaaaaaagaatccctcacacaccgcgccgaacgcaccgcaagcttccggtgcgttcggcacggtgtataagggacggcc

SEQ ID NO: 11 - DNA (nucleic acid), 74 bp; single-stranded, linear
gcgtgatgagttgcacggtggaggtgagggaagcttgcttcgcctccaccgtgcaattcatcgcgcagtttttt

SEQ ID NO: 12 - DNA (nucleic acid), 82 bp; single-stranded, linear
aattaaaaaactgcgcgatgaattgcacggtggaggcgaagcaagcttccctcacctccaccgtgcaactcatcacgcggcc

SEQ ID NO: 13 - DNA (nucleic acid), 74 bp; single-stranded, linear
ggatggaggagatctcgctggcagggattgaagcttgagtctctgccggcgagatctcctccgtcctgtttttt

SEQ ID NO: 14 - DNA (nucleic acid), 82 bp; single-stranded, linear
aattaaaaaacaggacggaggagatctcgccggcagagactcaagcttcaatccctgccagcgagatctcctccatccggcc

SEQ ID NO: 15 - DNA (nucleic acid), 72 bp; single-stranded, linear
tcaccacataattacctttcttttcctcgaagcttggagggaaaggaaggtagttgtgtggtgatctttttt

SEQ ID NO: 16 - DNA (nucleic acid), 80 bp; single-stranded, linear
aattaaaaaagatcaccacacaactaccttcctttccctccaagcttcgaggaaaagaaaggtaattatgtggtgaggcc

SEQ ID NO: 17 - DNA (nucleic acid), 72 bp; single-stranded, linear
tgtcaccacataattacctttcttttccgaagcttggggaaaggaaggtagttgtgtggtgacatctttttt

SEQ ID NO: 18 - DNA (nucleic acid), 80 bp; single-stranded, linear
aattaaaaaagatgtcaccacacaactaccttcctttccccaagcttcggaaaagaaaggtaattatgtggtgacaggcc

SEQ ID NO: 19 - DNA (nucleic acid), 72 bp; single-stranded, linear
ctgtcaccacataattacctttcttttcgaagcttgggaaaggaaggtagttgtgtggtgacagtctttttt

SEQ ID NO: 20 - DNA (nucleic acid), 80 bp; single-stranded, linear
aattaaaaaagactgtcaccacacaactaccttcctttcccaagcttcgaaaagaaaggtaattatgtggtgacagggcc